(19) United States
(12) Patent Application Publication
(10) Pub. No.: US 2021/0106459 A1
(43) Pub. Date: Apr. 15, 2021

(54) Systems and Methods For Improved Thermally Controlled Therapeutic Devices

(71) Applicants: Louie J. Caruso, Glendale, AZ (US); David Sardella, Glendale, AZ (US)

(72) Inventors: Louie J. Caruso, Glendale, AZ (US); David Sardella, Glendale, AZ (US)

(21) Appl. No.: 17/011,301

(22) Filed: Sep. 3, 2020

Related U.S. Application Data
(60) Provisional application No. 62/915,498, filed on Oct. 15, 2019.

(51) Int. Cl. A61F 7/00; A61F 7/02; A61F 7/08; G06F 3/01

(52) U.S. Cl. CPC A61F 7/007; A61F 7/02; A61F 7/08; G06F 3/011; A61F 2007/0094; A61F 2007/0075; A61F 2007/0003; A61F 2007/0039; A61F 2007/0032; A61F 2007/0024; A61F 2007/0023; A61F 2007/0077

(57) ABSTRACT

A wearable, compact therapeutic thermal control system includes a therapeutic component attached to the body of the patient, and a thermal control module is fluidically coupled to the therapeutic component. The thermal module includes a power supply; a controller; a wireless interface communicatively coupled to the controller; an electrothermal device, and electrical terminals communicatively coupled to the controller; a heatsink/fan subassembly thermally coupled to the first side of electrothermal device; a heat exchanger block thermally coupled to the second side of the electrothermal device; and a pump device configured to cause flow of a working fluid through the heat exchanger block and the therapeutic component. The electrothermal device has a first mode in which heat energy is removed from the working fluid via the heat exchanger block, and a second mode in which heat energy is supplied to the working fluid.

### CROSS-REFERENCE TO RELATED APPLICATIONS

This application claims priority to U.S. Provisional Patent Application No. 62/915,498, entitled “Mobile Cold Therapy,” filed Oct. 15, 2019, the entire contents of which are hereby incorporated by reference.

### TECHNICAL FIELD

The present invention relates, generally, to therapeutic techniques for heating and cooling and, more particularly, to compact systems for heating, cooling, temperature cycling, and otherwise treating patients using orthopedic devices and the like.

### BACKGROUND

There are a number of therapeutic treatments that involve heating, cooling, or otherwise providing local stimulation using a pad or other orthopedic device thermally and/or mechanically coupled to the body of a patient. Cold therapy (or “cryotherapy”) may be used, for example, to treat pulled muscles, osteoarthritis, gout, tendonitis, joint pain, sprains, inflammation, and the pain and swelling resulting from surgery and other medical procedures. Such treatment might also be used in connection with burn victims. Cold therapy works by vasoconstriction of the treated area, and plays an important part in the standard “RICE” formula for injury treatment—i.e.: rest, ice, compression, and elevation, or “PRICE”—protection, rest, ice, compression, and elevation.

Heat therapy, on the other hand, generally involves increasing blood flow through vasodilation. That is, the treated tissue is heated to some predetermined level above body temperature. Such therapy may be used, for example, to relax and sooth muscles and to heal damaged tissue. Heat energy may be applied via any combination of conduction, convection, and radiation heat transfer, and may involve either “dry” or “wet” heating.

In addition to simply cooling or heating a region of the body, it is sometimes advantageous to apply temperature cycling or “contrast therapy”—i.e., cycling through alternating cooling and heating phases. Furthermore, compression and or vibratory excitation of a region may also be used for therapeutic purposes.

Despite recent advances in electronics and the miniaturization of electromechanical systems, currently known therapeutic cooling and heating devices are unsatisfactory in a number of respects.

For example, many treatments involve heating or cooling the affected area for a very short duration, and include significant downtime between applications. Such methods include, for example, ice packs, coolant sprays, ice baths, and heating pads. In addition, these approaches do not provide any form of thermal control (that is, the thermal conditions of the tissue never reach equilibrium), and generally require the patient to hold the treatment device in place or awkwardly fix the device to the body in an ad hoc manner.

While more advanced, automated cooling and heating systems are available, they are not portable and/or fully ambulatory. That is, such systems tend to be large and require the patient to be tethered to the machine, typically while seated or in a reclined position. For example, one popular form of cold therapy includes the use of a large container of ice water, a water pump, and many yards of tubing strapped to the leg, arm, or other body part of the patient via a pad or orthopedic device.

Not only is this form of therapy an inconvenience, it also fails to provide any means of temperature control beyond changing the flow rate of the working fluid. Furthermore, in presently known systems it is difficult for the patient to control her own therapy in a convenient manner. An attendant or caregiver must perform the onerous tasks of replacing large bags of ice, draining water, and otherwise managing the mechanical and thermal behavior of the system. This lack of control is more than an inconvenience; it can be damaging to the patient, as excessively low (or high) temperatures applied for extended periods of time can result in tissue damage.

Finally, prior art systems fail to appreciate the value of providing both heating and cooling (and possibly other forms of therapy) in a single unit. Systems and methods are therefore needed for overcoming these and other limitations of the prior art.

### SUMMARY OF THE INVENTION

Systems and methods in accordance with the present invention address the above limitations of prior art cooling/heating systems by providing, inter alia, i) a compact, wearable thermal module providing both cooling and heating functionality (and optionally compression and muscle stimulation) via an orthopedic component thermally coupled to the patient either directly (physical contact) or indirectly (via thermal radiation and/or imposed air movement, such as might be used with burn victims); ii) a thermal module as above including a wireless interface configured to communicate with a mobile device, wherein operation of the thermal module is controllable by a user via the mobile device; iii) a thermal module and orthopedic device as above, further configured to provide therapeutic pressure and/or vibratory stimulation; iv) a thermal system as above, wherein the orthopedic component is configured to be thermally coupled to the user's face; v) a thermal system as above, wherein the orthopedic component is a head-mounted display; vi) a thermal system as above, wherein the orthopedic component is configured to be secured to an equine patient; vii) a thermal system as above, wherein the orthopedic component is configured to be secured to a canine patient; viii) a thermal system as above, wherein the orthopedic component is configured as a motorcycle helmet including one or more cooling/heating surfaces coupled thereto; ix) a thermal module as above, further including one or more Peltier components in thermal communication with a heat-exchanger block; x) a thermal module as in the preceding, wherein the Peltier components comprise two Peltier devices mounted in parallel to the heat-exchanger block.

### BRIEF DESCRIPTION OF THE DRAWING FIGURES

The present invention will hereinafter be described in conjunction with the appended drawing figures, wherein like numerals denote like elements, and:

FIG. 1 is a conceptual overview of a mobile, wearable thermally controlled therapy system in accordance with various embodiments;

FIG. 2 is an isometric overview of a thermal module in accordance with one embodiment, in which the Peltier components are in direct contact with the heatsink;

FIG. 3 is an isometric overview of a thermal module in accordance with an alternate embodiment, in which the Peltier components are thermally coupled to the Peltier components via a heat pipe assembly,

FIG. 4 is an isometric overview of a finned heatsink in accordance with one embodiment;

FIGS. 5A and 5B illustrate isometric and cross-sectional views of a heat-exchange block in accordance with one embodiment;

FIG. 6 illustrates a miniature pump device in accordance with one embodiment;

FIG. 7 illustrates a Peltier component in accordance with one embodiment;

FIGS. 8A-8C illustrate the use of the systems of the present invention in connection with a facial mask or pad;

FIGS. 9A-9C illustrate the use of systems of the present invention in connection with a head mounted display (e.g., mixed reality headset) and an associated facial mask;

FIGS. 10A-10C illustrate the use of systems of the present invention in connection with a leg wrap;

FIGS. 11A-11C illustrate the use of systems of the present invention in connection with and arm/elbow wrap;

FIGS. 12A-12C illustrate the use of systems of the present invention in connection with a motorcycle helmet and associated neck pad;

FIG. 13 illustrates the use of systems in accordance with the present invention for equine therapy;

FIGS. 14A-14C illustrate the use of systems of the present invention in connection with a canine therapeutic pad;

FIG. 15 illustrates a non-limiting, example user interface that a user may employ to provide thermal control of the module in accordance with various embodiments.

FIG. 16 is a schematic depiction of a thermo-electric pre-cooler in accordance with one embodiment; and

FIG. 17 illustrates a waist-worn embodiment of the present invention.

### DETAILED DESCRIPTION OF PREFERRED EXEMPLARY EMBODIMENTS

The present subject matter generally relates to systems and methods for a compact, wearable, and portable therapeutic thermal control system capable of both heating and cooling that can be controlled by the user via an intuitive user interface provided on an external mobile device. The compact nature of the system derives, in part, from the efficient use of efficient but small thermoelectric device (e.g., Peltier devices) in combination with one or more fans, heatsinks, and heat-exchanger components as described in further detail below.

As a preliminary matter, it will be understood that the following detailed description is merely exemplary in nature and is not intended to limit the inventions or the application and uses of the inventions described herein. Furthermore, there is no intention to be bound by any theory presented in the preceding background or the following detailed description. In the interest of brevity, conventional techniques and components related to heat transfer, pumps, cooling devices, and control systems may not be described in detail herein.

General Overview

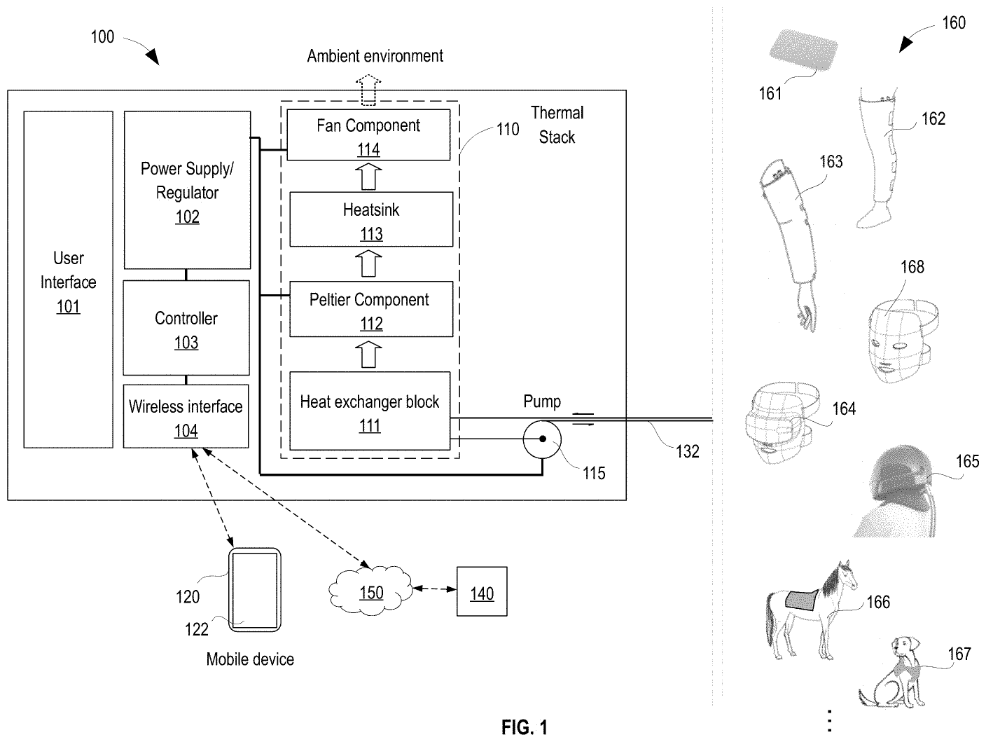

Referring first to FIG. 1, the present invention may be implemented in the context of a system 100 that includes a thermal control module (or simply “module”) 100 in thermal communication (e.g., via tubing segment 132) with an orthopedic component selected from a variety of such components 160, as described in further detail below.

Module 100 generally includes a power supply and regulator module 102 (e.g., one or more removable LiPo, Li-ion, NiCd, or NiMH rechargeable battery packs, or a battery pack external to module 100), a controller 103, an optional user interface 101 (including manually actuatable switches, dials or the like incorporated into the housing of module 100), a wireless interface 104, and a thermal stack 110 comprising a number of components in thermal communication—i.e.: a heat-exchanger block 111 (which may function as a “cooling block” or “heating block” depending upon the selected mode), a Peltier or other thermoelectric device 112, a heatsink 113, and a fan component 114. Module 100 includes a pump 115 configured, via suitable tubing or other fluid pathway, to control the flow of a working fluid between block 111 and the orthopedic component 160 as shown. The heat transfer fluid (or “working fluid”) may vary depending upon application, from water to various water/coolant mixtures (such as glycol/water, glycerin, hydrocarbon oils, phase-change fluids, or the like).

In various embodiments, module 100 is wirelessly coupled (e.g., via Bluetooth LE, WiFi, or any other suitable protocol implemented by wireless interface 104) to mobile device 120. Mobile device 120 may include, without limitation, a smartphone, a tablet computer, a laptop computer, or any other portable computing device that includes a display screen 122 and is capable of communicating with module 100 via wireless interface 104. An application is provided (e.g., downloadable and installed via an app store) for mobile device 120 that allows a user (or other individual) to control the setpoint temperature and other operational behavior of module 100 in a convenient and intuitive manner.

Module 100 may also be configured to communicate, using wireless interface 104, with one or more external servers 140 via a network (e.g., the Internet) 150. This functionality allows a user remotely located from the patient to modify the applied treatment by, for example, changing the mode of module 100 (i.e., heating or cooling) as well as the setpoint temperature. In addition, server 140 may be used to store preferences and configuration information associated with module 100.

Controller 103 is communicatively coupled to power supply 102 as well as fan component 114, Peltier component 112, and pump 115. Suitable software, hardware, and/or firmware is incorporated into controller 103 to achieve the functionality described herein. It will be appreciated that FIG. 1, as a conceptual block diagram, is not intended to be limiting, and that a variety of semiconductor device architectures may be used to implement embodiments of the present invention.

The various components of thermal stack 110 (i.e., fan 114, heatsink 113, Peltier 112, and block 111) are illustrated in a manner that is agnostic as to what form of thermal connectivity exists between those components. As described in further detail below, the components are generally connected via thermally conductive paths, but might also be coupled via convection/radiation paths. In addition, heat pipes, heat spreaders, and other structures for distributing heat energy may be used within thermal stack 110, but in the interest of simplicity are not illustrated in FIG. 1.

As mentioned above, a wide variety of pads and other orthopedic devices may be used in connection with the system of FIG. 1. In this regard, the phrases “orthopedic component” and “orthopedic device” are used in a non-limiting sense to encompass any device or structure that is worn by, or secured to, the patient and which includes one or more surfaces in thermal communication with the working fluid conveyed via tubing 132 (e.g., via a serpentine fluid pathway incorporated into its structure). In the illustrated embodiment, for example, the orthopedic components 160 are shown to include a pad 161, a leg brace 162, an arm brace 153, a facial pad 163, a head-mounted display 164, a motorcycle helmet assembly 165, and various veterinary applications, such as an equine pad 166 and canine pad 167. In the interest of clarity, tubing 132 has not been extended to the various components 160, but it will be understood that such tubing would typically be removably or non-removably attached, depending upon the embodiment. Furthermore, as will be apparent, the orthopedic components 160 are not limited to use on human beings, but in fact may be adapted and configured to be worn by any mammal or other member of the animal kingdom for which localized cooling or heating is desirable.

With respect to controller 103, wireless interface 104, power supply 102, and/or user interface 101, the terms “module” or “controller” refer to any suitable hardware, software, firmware, electronic control component, processing logic, and/or processor device, individually or in any combination. Such components include, without limitation: application specific integrated circuits (ASICs), field-programmable gate-arrays (FPGAs), electronic circuits, processors (shared, dedicated, or group) configured to execute one or more software or firmware programs, a combinational logic circuit, and/or other suitable components that provide the described functionality.

In one embodiment, power supply 102 is selected such that its capacity is sufficient to operate module 100 for approximately 20-60 minutes on a single charge. In one embodiment, the power supply 102 comprises a set of quick-change, rechargeable batteries that can be easily removed and replaced by the user to extend the duration that the system operates during a given session.

General Operation

With continued reference to the conceptual block diagram of FIG. 1, the operation of module 100 will vary depending upon the mode that is currently active (i.e., heating mode vs. cooling mode), which itself is dependent upon the polarity of the voltage applied to Peltier component 112.

In cooling mode, operation of the system proceeds as follows: (1) the therapeutic component 160 is fixed to, worn, or otherwise secured to the patient; (2) a cooling temperature setpoint is selected via an application provided by mobile device 120; (3) the requested setpoint is transmitted to module 100 via wireless interface 104 (alternatively, the user may make a selection using the optional user interface 101 provided within module 100); (4) controller 103 adjusts the voltage applied to Peltier component 112, fan component 114, and pump 115 in accordance a standard control system algorithm (e.g., a PID controller) in order to reach the desired temperature set point; (5) heat energy from the patient is transferred, via the working fluid, to block 111; (6) that heat energy is conducted to the low side (cooler side) surface of Peltier component 112; (7) heat energy from the high side of Peltier component 112 is transferred to heatsink 113, which is then ejected to the ambient environment via fan component 114. The controller modulates the fluid temperature in accordance with a target (e.g., approximately 30.0-40.0° F. in the cooling mode) based on the target temperature selected by the user.

During the heating mode, the thermal conditions are largely reversed by reversing the polarity. That is, power is applied to Peltier Component 112 such that the surface in thermal communication with block 111 becomes the hotter surface, and that generated heat energy is transferred to the patient via pump 115 and the working fluid within tubing 132.

Control Module: Example Physical Configurations

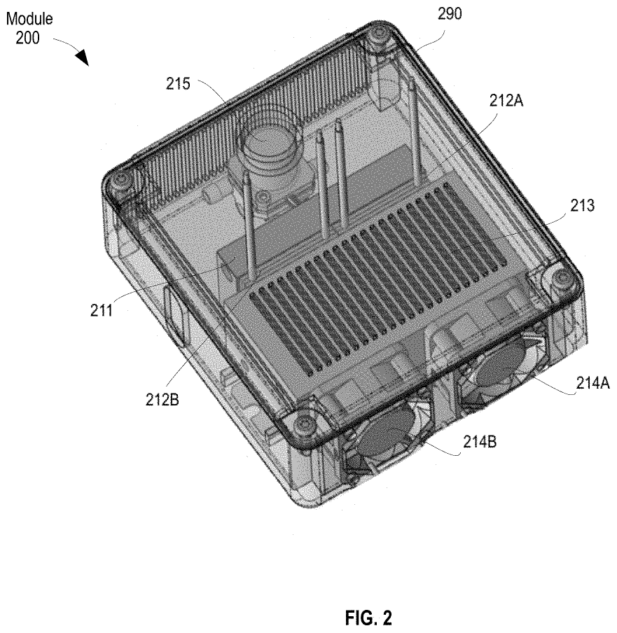

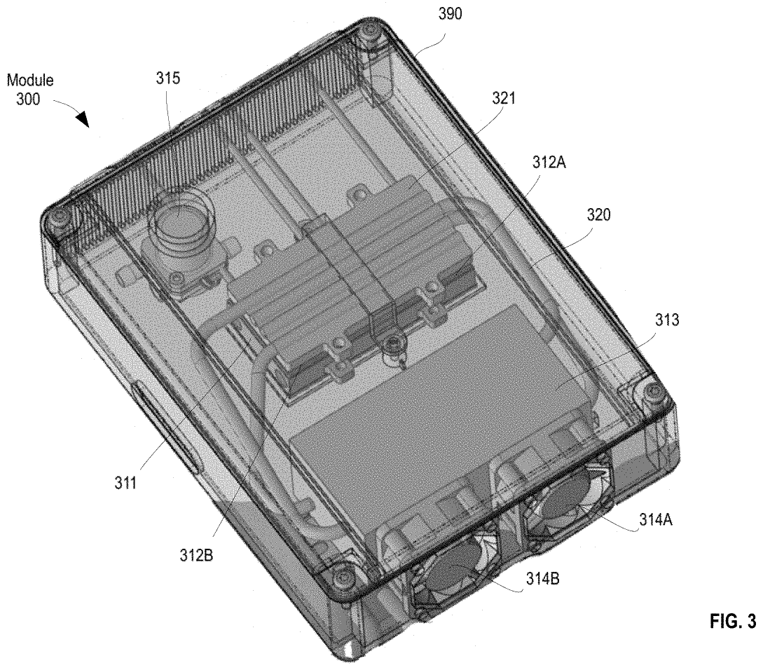

Modules in accordance with the present invention may be implemented in a compact form with a variety of footprints, but preferably has a weight and size that is convenient for an ambulatory user. In that regard, FIGS. 2 and 3 illustrate just 2 example embodiments, which are not intended to be limiting in any way.

First, FIG. 2 is an isometric overview of a thermal module in accordance with one embodiment, in which the Peltier components are in direct contact with the heatsink. More particularly, module 200 includes a housing 290 containing a pair of compact fans 214A and 214B, a heatsink 213, a pair of thermoelectric (Peltier) devices 212A and 212B, a heat-exchanger block 211, and a pump 215. For simplicity, pump 215 is illustrated without its associated electrical and hydraulic connections.

It can be seen that the embodiment illustrated in FIG. 2 is particularly compact, given that the two Peltier devices 212A and 212B are situated side-by-side, sandwiched between and in intimate contact with heat-exchanger block 211 and heatsink 213. Thus, Peltier devices 212A and 212B, when property activated (i.e., with the same relative polarity) operate in parallel to cool a larger area of heat-exchanger block 211 efficiently.

FIG. 3, on the other hand, is an isometric overview of a thermal module in accordance with an alternate embodiment, in which the Peltier components are thermally coupled to the Peltier components via a heat pipe assembly, that is, module 300 includes, as before, a pump 315, a heatsink 313, a pair of fans 314A and 314B, a heat-exchanger block 311, and a pair of Peltier components 312A and 312B. It also includes a set of heat-pipes or a single contiguous heat pipe 320 that is thermally coupled to both heatsink 313 and a second heat exchanger block 321—which itself is in thermal contact with the Peltier components 312A and 312B all within a housing 390.

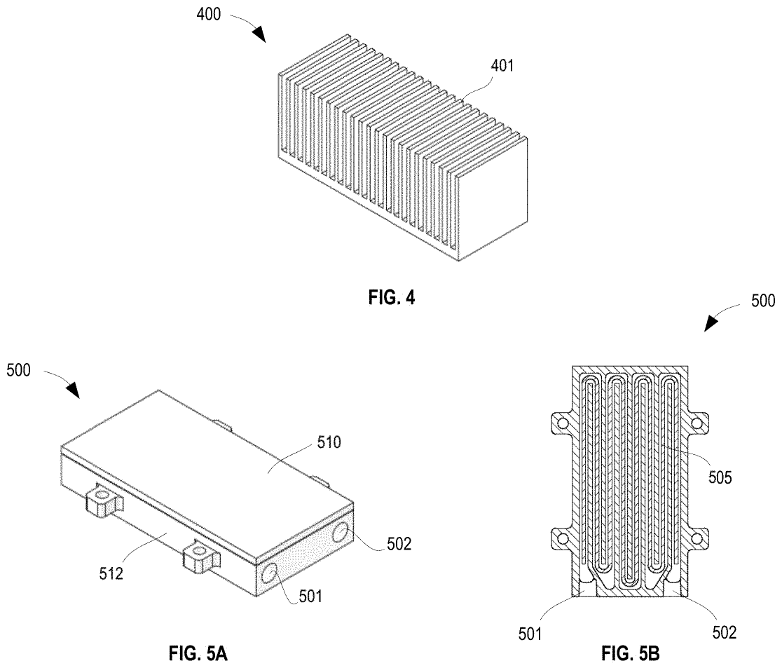

In the interest of providing efficient and compact heating/cooling, a variety of small form-factor components may be used in connection with the present invention. FIGS. 4-7 show just a few non-limiting examples.

FIG. 4 is an isometric overview of an example finned heatsink 400 in accordance with one embodiment, including a series of thin fins 401 in a compact structure. In one embodiment, for example, the bottom surface (the “heat source area”) is a rectangular area approximately 3″×1.5″, with a fin height about approximately 1.5-2.0″, and a fin width of about 1.5-1.7″. Heatsink 400 may be manufactured from copper or any other suitable material with a high thermal conductivity.

FIGS. 5A and 5B illustrate isometric and cross-sectional views of a heat-exchange block 500 in accordance with one embodiment. As shown, block 500 includes a thermally conductive bottom portion 512 and a top portion 510. Bottom portion 512 includes an inlet 501 and outlet 502 which, as shown in FIG. 5B, are connected by a fine pitch serpentine pattern 505 for providing efficient forced convection heat transfer when the working fluid flows from inlet 501 to outlet 502. In one embodiment, block 500 is a machined copper component e.g., copper alloy 110 having a length of approximately 3-4 inches, a width of approximately 1-2 inches, and a thickness of approximately 0.4-0.6 inches). The top portion 510 and bottom portion 512 may be solder brazed together or otherwise permanently joined. Inlet and output ports 501 and 502 may have an inner diameter of approximately 0.15-0.20 inches.

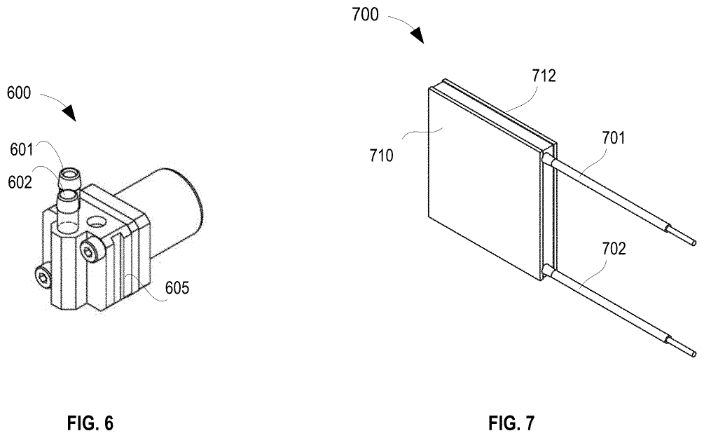

FIG. 6 illustrates a miniature pump device (or simply “pump”) 600 in accordance with one embodiment. As shown, pump 600 includes a body portion 605, an inlet 601, and an outlet 602. In one embodiment, pump 600 is approximately 0.5″×0.5″×1.0″ in, and has a flow rate in the range of approximately 90-400 mLpm (driven at approximately 0.5 W to 1.8 W).

FIG. 7 illustrates a Peltier component 700 in accordance with one embodiment. In this embodiment, component 700 includes opposing sides 710 and 712, which function as “hot” and “cold” sides, depending upon the direction of current flow through leads 701 and 702. Peltier component 700, in the illustrated embodiment, has a thickness of approximately 0.1 to 0.2″, and a surface region of about 0.5 to 1.25″ on side. A variety of materials may be used for Peltier component 700, including, for example, 96% Al₂O₃ or the like operating at approximately 0 to 8.0 VDC (corresponding to approximately 0 to 40 W of heat energy transferred).

Orthopedic Components/Application Examples

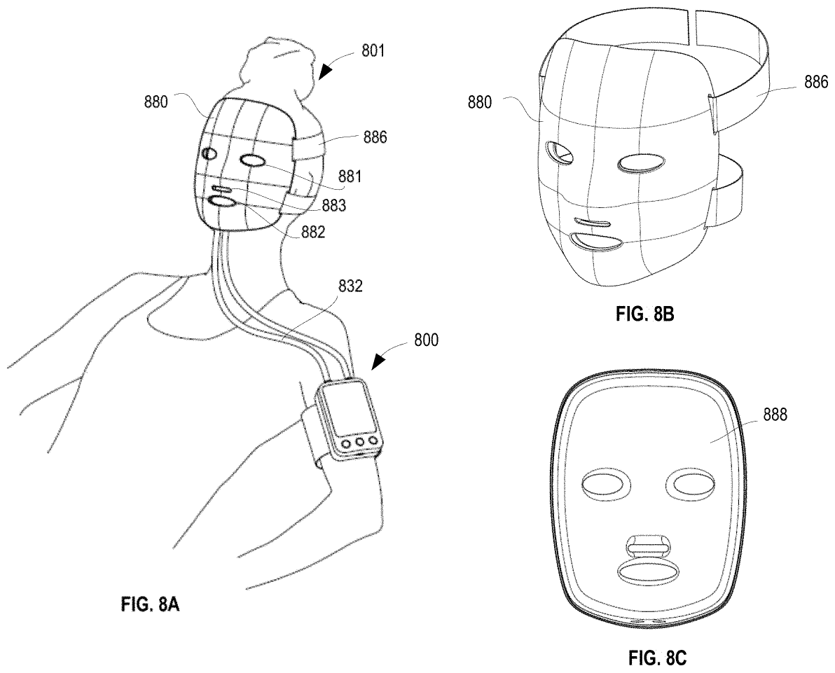

As mentioned above, the present invention may be used in connection with a wide range of orthopedic components and in a variety of contexts—both for humans and non-human animals. In that regard, FIGS. 8-14 show a number of prominent examples. It will be appreciated that nothing in these figures is intended to be limiting as to orthopedic devices, connection methods, anatomical treatment locations, module connection methods, or the like.

FIGS. 8A-8C illustrate the use of the system of the present invention in connection with a facial mask or pad, which may operate in either a heating, cooling, or contrast mode. User 801 is shown wearing a facial mask 880 secured via a rear strap assembly 886. Mask 880, in this embodiment, includes eye openings 881, a nose opening 883, and a mouth opening 882. Mask 880 is thermally coupled, via tubing 832, to module 800, which may be implemented in accordance with the various modules described above. In this embodiment, as shown, module 800 is configured to attach to the user's arm. This allows user 801 to move around with very little constraints on her activity while still experiencing the beneficial effects of the system. FIG. 8B illustrates the example rear strap assembly 886 in greater detail (which might incorporate, for example, a VELCRO-like attachment method to join the strap segments at the back of the head). FIG. 8C illustrates the inner surface of mask 880, including the surface 888 that is thermally coupled to the working flu flowing within mask 880. In some embodiments, surface 888 is in contact with the user's face; in others, it is not in direct contact, but heats or cools the user's face via convection and/or radiation heat transfer.

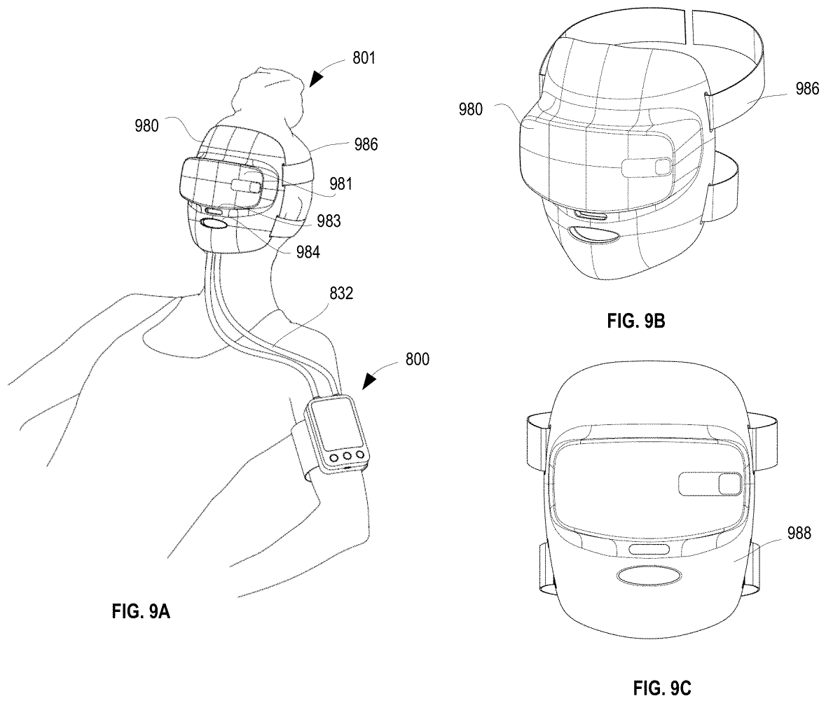

FIGS. 9A-9C illustrate the use of systems of the present invention in connection with a head mounted display (e.g., mixed reality headset) and an associated facial mask. This is a variation of the embodiment illustrated in FIGS. 8A-8C, wherein mask 980 includes a mounting strap 986, a nose opening 983, a mouth opening 984, and an integrated display screen that is viewable by the user (e.g., when playing a video game or otherwise taking part in a virtual-reality or mixed-reality experience. As shown in FIG. 9C, the inner surface 988 is thermally coupled to the working fluid being pumped through mask 980.

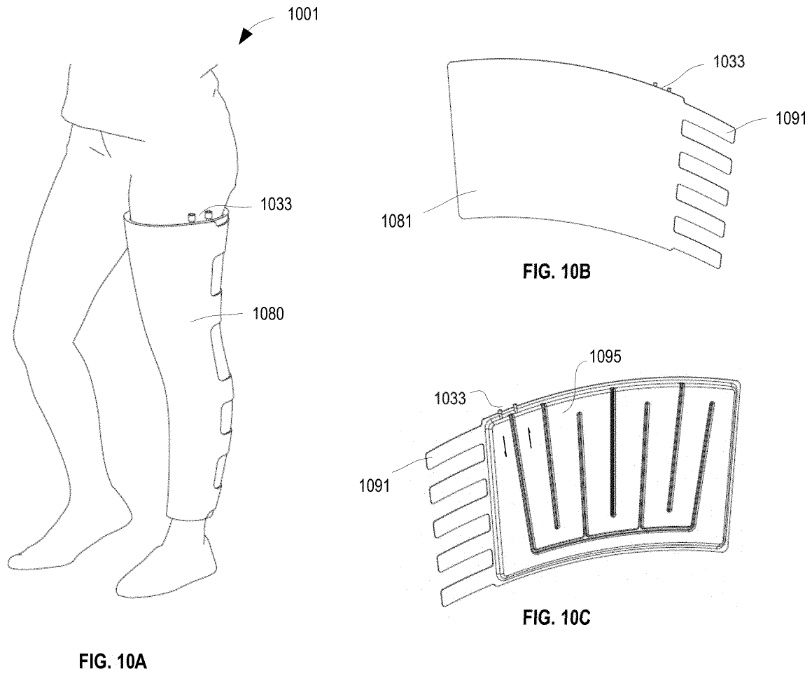

FIGS. 10A-10C illustrate the use of systems of the present invention in connection with a leg wrap. That is, a wrap component 1080 having a pair of inlet/outlet ports 1033 incorporated therein is shown wrapped around the leg of a user 1001. Wrap component 1080 includes a cooling/heating surface 1081 and a series of attachment straps 1091 that, as with the facial mask embodiment shown above, might incorporate a VELCRO-like attachment method. FIG. 10C illustrates, in a cut-away view, the internal serpentine pattern 1095 through which the working fluid travels to and from input/output ports 1033.

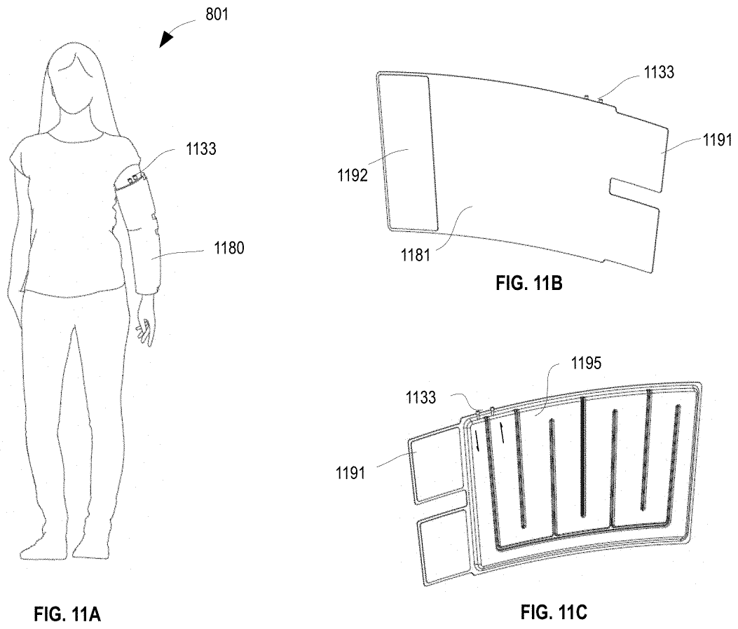

FIGS. 11A-11C illustrate the use of systems of the present invention in connection with and arm/elbow wrap. As with the embodiment shown above, the wrap component 1180 includes a pair of inlet/outlet ports 1133 and is wrapped around the arm of a user. Wrap component 1080 includes a cooling/heating surface 1181 and a pair of straps 1191 that attach to an opposing surface 1192 when worn by user 401. FIG. 10C illustrates, in a cut-away view, the internal serpentine pattern 1195 through which the working fluid travels to and from input/output ports 1133.

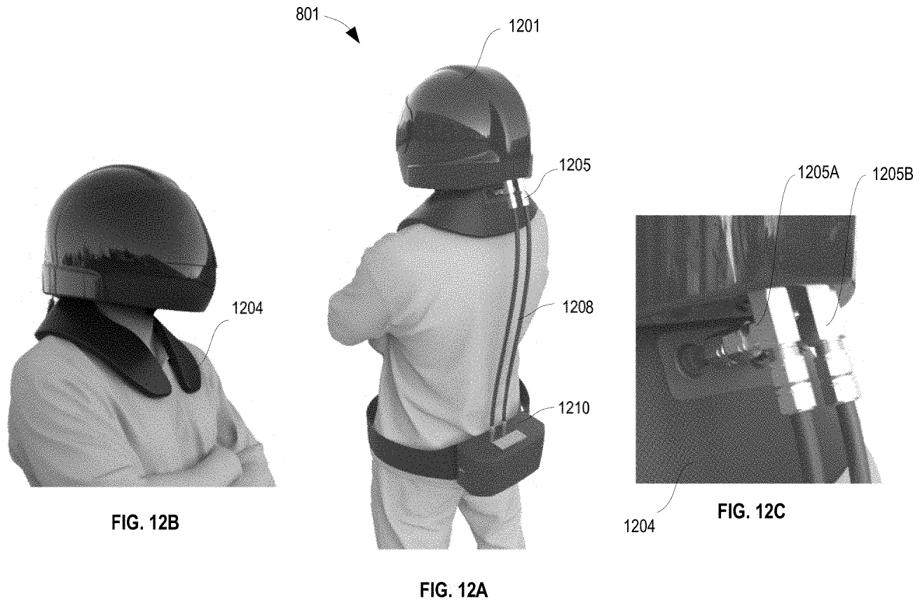

FIGS. 12A-12C illustrate the use of systems of the present invention in connection with a motorcycle helmet and associated neck, shoulder, and upper chest pad. The purpose of this embodiment is primarily to provide comfort to riders on long journeys and/or inclement weather. In this embodiment, as shown in FIG. 12A, the central module 1210 is attached via a belt to the rider's hip, and includes tubing 1208 extending to fittings 1205 attached to a pad within helmet 1201 as well as a neck pad 1204. As shown in FIG. 12C, in one embodiment, the fittings include a inlet fitting 1205A and a output fitting 1205B that together attach (using, for example, a quick-release attachment mechanism) to both helmet 1201 and neck pad 1204. While not illustrated in FIGS. 12A-12C, helmet 1201 and neck pad 1204 include respective internal pathways through which the working fluid can flow, providing the required cooling and/or heating. Cooling/heating may be provided via contact means, or via a fan or other forced area assembly within the helmet itself.

As shown in FIG. 12A, module 1210 may include a display and one or more user interface elements to allow direct control of the system. In other embodiments, module 1210 may wirelessly interface with a cellular phone or display system integrated into the motorcycle being operated by the user.

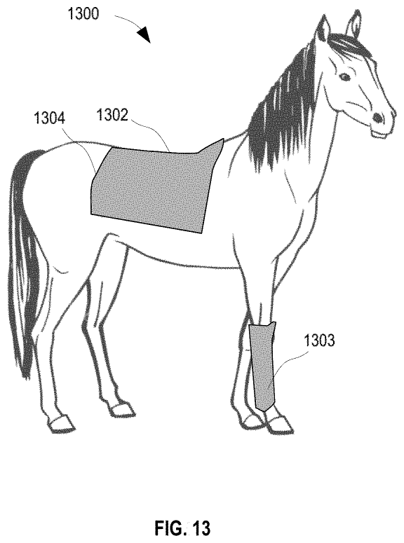

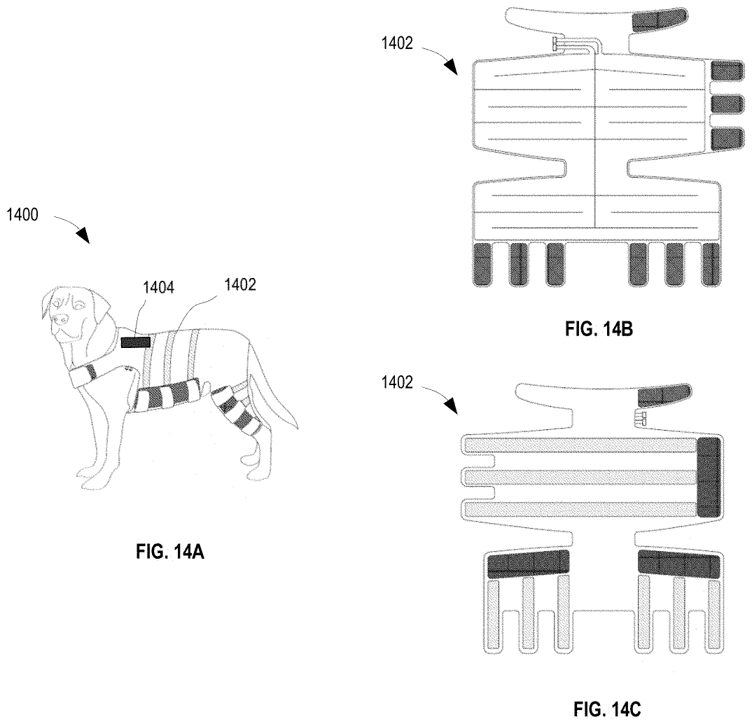

As mentioned briefly above, the present invention is not limited to humans. There are a lot of instances in which a dog, horse, cat, or other non-human animal may benefit from heating and/or cooling applied to the body. FIGS. 13 and 14 illustrate, respectively, just two examples: equine therapeutic pads and canine therapeutic pads. More particularly, FIG. 13 illustrates a horse 1300 on which a back therapeutic pad 1302 and leg therapeutic pad 1303 has been fitted. A module (not illustrated) may be attached anywhere along an edge 1304 of pad 1302. In such embodiments, the module/power supply may be incorporated into pad 1302 itself. It may also be secured the bridle, saddle, leg, or other convenient component. Similarly, FIG. 14A illustrates a dog 1400 wearing a therapeutic pad 1402 bearing a control module 1404. FIGS. 14B and 14C illustrate top and bottom views of pad 1402, wherein FIG. 14B shows the serpentine path through which the working fluid travels. In both of these embodiments, a human user may adjust the cooling/heating mode and setpoint temperature of pads 1302 and 1402 via a mobile computing device and/or via an external network, as described above in conjunction with FIG. 1.

User Interface Example

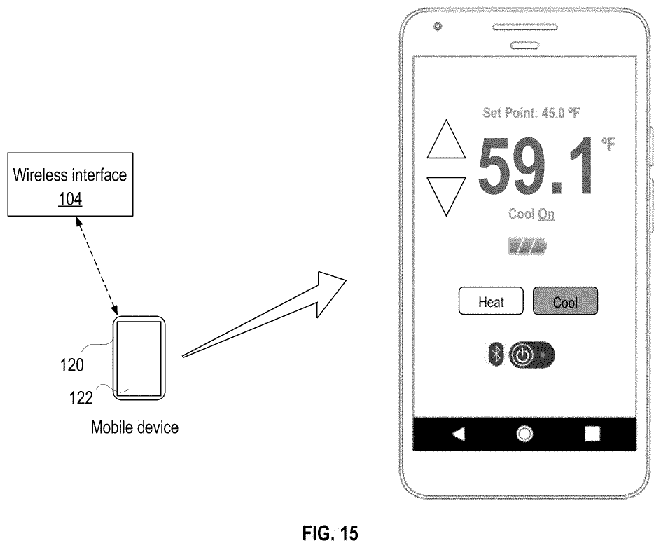

FIG. 15 illustrates an example, non-limiting user interface that a user may employ to provide thermal control of the module in accordance with various embodiments. That is, FIG. 15 illustrates the communication of mobile device 120 with wireless interface 104. Touchscreen 122 of device 120 is used to present the user interface, which in this embodiment includes a variety of user interface icons presented in one, intuitive screen. That is, the user interface allows a user to turn the system on and off, select a mode (i.e., “heat” or “cool” mode), and then adjust a set point temperature within a predefined range for that mode. The display also shows the current temperature, the status of the battery, and status of the wireless connection (in this case, a Bluetooth connection). It will be appreciated that the interface illustrated in FIG. 15 merely presents one example, and is not intended to be limiting in terms of functionality or interface elements.

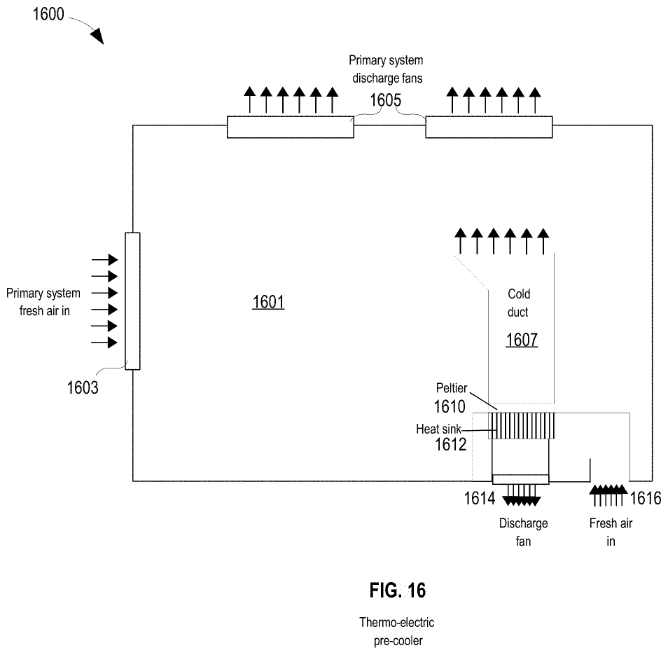

FIG. 16 illustrates an example thermo-electric pre-cooler 1600 that may be used in connection with the present invention to improve efficiency in some circumstances. Briefly, the module 100 described above in FIG. 1 may be incorporated into or otherwise used on conjunction with the pre-cooler of FIG. 16, which includes an inner environment 1601 (in which one or more components of module 100 may be placed), a primary vent 1603 for receiving fresh air, a primary discharge fan or fans 1605, and a subsystem for pre-cooling a portion of fresh air. This subsystem includes a fresh air inlet 1616, a discharge fan 1614, a heatsink 1612, and a Peltier or other thermo-electric device 1610. Fresh air from inlet 1616 passes over or otherwise interacts with Peltier device 1610 to provide air below outside ambient temperature, which is then fluidically coupled to the rest of the system via cold duct 1607. The primary function of pre-cooler 1600 is to provide cooled air to the thermal stack 110 of FIG. 1. Peltier 1610 may be powered using the power supply of FIG. 1 or via a separate power source.

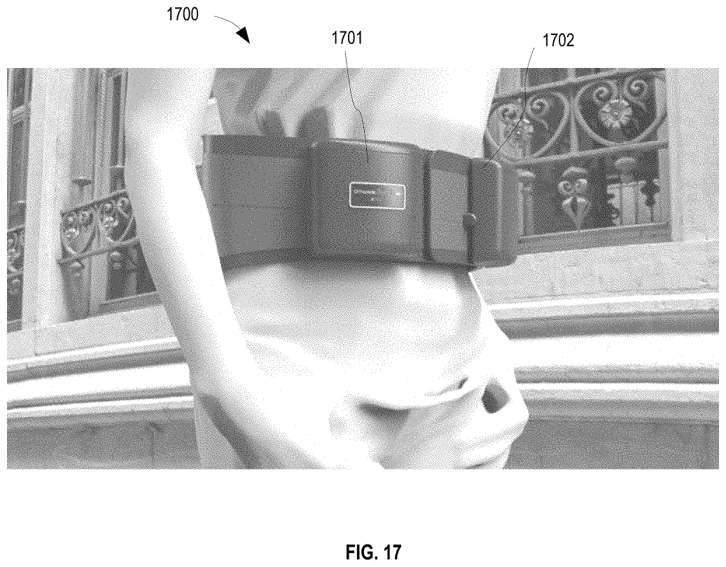

FIG. 17 illustrates a particular embodiment of a module of the present invention that is worn at the waist. That is, the system includes a belt assembly 1700 as shown, which can be releasably attached to the waist of the user. In the illustrated embodiment, assembly 1700 includes a battery pack or other power supply 1702 and a connected thermal control module 1701 as described above. While not shown in FIG. 17, module 1701 is suitably connected, fluidically, to a therapeutic device fixed to the body of the user.

Embodiments of the above heating/cooling device are described in terms of functional and/or logical block components and various processing steps. It should be appreciated that such block components may be realized by any number of hardware, software, and/or firmware components configured to perform the specified functions. For example, an embodiment of the present disclosure may employ various integrated circuit components, e.g., memory elements, digital signal processing elements, logic elements, look-up tables, or the like, which may carry out a variety of functions under the control of one or more microprocessors or other control devices.

In addition, the various functional modules described herein (i.e., the cloud computing server and heating/cooling algorithm of FIG. 1) may be implemented entirely or in part using a machine learning or predictive analytics model. In this regard, the phrase “machine learning” model is used without loss of generality to refer to any result of an analysis that is designed to make some form of prediction, such as predicting the state of a response variable, clustering patients, determining association rules, and performing anomaly detection. Thus, for example, the term “machine learning” refers to models that undergo supervised, unsupervised, semi-supervised, and/or reinforcement learning. Such models may perform classification (e.g., binary or multiclass classification), regression, clustering, dimensionality reduction, and/or such tasks. Examples of such models include, without limitation, artificial neural networks (ANN) (such as a recurrent neural networks (RNN) and convolutional neural network (CNN)), decision tree models (such as classification and regression trees (CART)), ensemble learning models (such as boosting, bootstrapped aggregation, gradient boosting machines, and random forests), Bayesian network models (e.g., naive Bayes), principal component analysis (PCA), support vector machines (SVM), clustering models (such as K-nearest-neighbor, K-means, expectation maximization, hierarchical clustering, etc.), linear discriminant analysis models.

Any of the data generated by system 100 (e.g., user configuration data or health data) may be stored and handled in a secure fashion (i.e., with respect to confidentiality, integrity, and availability). For example, a variety of symmetrical and/or asymmetrical encryption schemes and standards may be employed to securely handle the therapeutic data at rest (e.g., in system 100) and in motion (e.g., when being transferred between the various modules illustrated above). Without limiting the foregoing, such encryption standards and key-exchange protocols might include Triple Data Encryption Standard (3DES), Advanced Encryption Standard (AES) (such as AES-128, 192, or 256), Rivest-Shamir-Adelman (RSA), Twofish, RC4, RC5, RC6, Transport Layer Security (TLS), Diffie-Hellman key exchange, and Secure Sockets Layer (SSL).

In summary, what has been described are systems and methods for a compact, wearable, and portable therapeutic thermal control system capable of both heating and cooling being fully ambulatory if needed, that can be controlled by the user via an intuitive user interface provided on an external mobile device.

In accordance with one embodiment, a wearable, compact therapeutic thermal control system comprises: a therapeutic component configured to be removably attached to the body of a user, and a thermal control module fluidically coupled to the therapeutic component and configured to be worn by the user. The thermal module includes: a power supply; a controller; a wireless interface communicatively coupled to the controller; a wireless interface coupled to the controller; an electrothermal device having a first surface, a second surface, and electrical terminals communicatively coupled to the controller; a heatsink/fan subassembly thermally coupled to the first side of electrothermal device; a heat exchanger block thermally coupled to the second side of the electrothermal device; and a pump device configured to cause flow of a working fluid through the heat exchanger block and the therapeutic component; wherein the controller is configured to operate the electrothermal device in at least two modes: a first mode in which heat energy is removed from the working fluid via the heat exchanger block, and a second mode in which heat energy is supplied to the working fluid via the heat exchanger block; wherein the controller operates the electrothermal device in response to a command received from a mobile device communicatively coupled to the wireless interface.

In accordance with one embodiment, the controller is configured to operate the electrothermal device in a third, thermal contrast mode in which heat energy is alternately removed and supplied to the working fluid via the heat exchanger block.

In accordance with one embodiment, the therapeutic component is selected from the group consisting of cooling pads, leg braces, arm/elbow braces, back braces, face pads or off the face design, virtual reality headsets, and comfort pads, motorcycle helmets.

In accordance with one embodiment, the electrothermal device comprises a pair of Peltier components coupled in parallel to the heat exchanger block.

In accordance with one embodiment, the thermal control module is configured to be secured to at least one of: a user's arm, a user's waist, a user's leg, or a user's head.

A method of treating a patient using a wearable, compact therapeutic thermal control system, generally includes: removably attaching a therapeutic component to the body of the patient; providing a thermal control module fluidically coupled to the therapeutic component and configured to be removably fixed to the patient's body, the thermal module comprising: a power supply; a controller; a wireless interface communicatively coupled to the controller; a wireless interface coupled to the controller; an electrothermal device having a first surface, a second surface, and electrical terminals communicatively coupled to the controller; a heatsink/fan subassembly thermally coupled to the first side of electrothermal device; a heat exchanger block thermally coupled to the second side of the electrothermal device; and a pump device configured to cause flow of a working fluid through the heat exchanger block and the therapeutic component; selectively operating the electrothermal device in at least two modes: a first mode in which heat energy is removed from the working fluid via the heat exchanger block, and a second mode in which heat energy is supplied to the working fluid via the heat exchanger block; operating the electrothermal device in response to a command received from a mobile device communicatively coupled to the wireless interface.

In addition, those skilled in the art will appreciate that embodiments of the present disclosure may be practiced in conjunction with any number of systems, and that the systems described herein are merely exemplary embodiments of the present disclosure. Further, the connecting lines shown in the various figures contained herein are intended to represent example functional relationships and/or physical couplings between the various elements. It should be noted that many alternative or additional functional relationships or physical connections may be present in an embodiment of the present disclosure.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

1. A wearable, compact therapeutic thermal control system comprising:
a therapeutic component configured to be removably attached to the body of a user;
a thermal control module fluidically coupled to the therapeutic component and configured to be worn by the user, the thermal module comprising:
a power supply;
a controller;
a wireless interface communicatively coupled to the controller;
a wireless interface coupled to the controller;
an electrothermal device having a first surface, a second surface, and electrical terminals communicatively coupled to the controller;
a heatsink/fan subassembly thermally coupled to the first side of electrothermal device;
a heat exchanger block thermally coupled to the second side of the electrothermal device; and
a pump device configured to cause flow of a working fluid through the heat exchanger block and the therapeutic component;
wherein the controller is configured to operate the electrothermal device in at least two modes: a first mode in which heat energy is removed from the working fluid via the heat exchanger block, and a second mode in which heat energy is supplied to the working fluid via the heat exchanger block;
wherein the controller operates the electrothermal device in response to a command received from a mobile device communicatively coupled to the wireless interface.

2. The thermal control system of claim 1, wherein the controller is configured to operate the electrothermal device in a third, thermal contrast mode in which heat energy is alternately removed and supplied to the working fluid via the heat exchanger block.

3. The thermal control system of claim 1, wherein the therapeutic component is selected from the group consisting of cooling pads, leg braces, arm/elbow braces, back braces, face pads, virtual reality headsets, comfort pads, and motorcycle helmets.

4. The thermal control system of claim 1, wherein the thermal control module is configured to be secured to at least one of: a user's arm, a user's waist, a user's leg, or a user's head.

5. The thermal control system of claim 1, wherein the electrothermal device comprises at least one Peltier component.

6. The thermal control system of claim 1, wherein the electrothermal device comprises a pair of Peltier components coupled in parallel to the heat exchanger block.

7. The thermal control system of claim 1, wherein the therapeutic component is configured to be fixed to an anatomical feature of a non-human animal.

8. A method of treating a patient using a wearable, compact therapeutic thermal control system, the method including:
removably attaching a therapeutic component to the body of the patient;
providing a thermal control module fluidically coupled to the therapeutic component and configured to be removably fixed to the patient's body, the thermal module comprising:
a power supply;
a controller;
a wireless interface communicatively coupled to the controller;
a wireless interface coupled to the controller;
an electrothermal device having a first surface, a second surface, and electrical terminals communicatively coupled to the controller;
a heatsink/fan subassembly thermally coupled to the first side of electrothermal device;
a heat exchanger block thermally coupled to the second side of the electrothermal device; and
a pump device configured to cause flow of a working fluid through the heat exchanger block and the therapeutic component;
selectively operating the electrothermal device in at least two modes: a first mode in which heat energy is removed from the working fluid via the heat exchanger block, and a second mode in which heat energy is supplied to the working fluid via the heat exchanger block;
operating the electrothermal device in response to a command received from a mobile device communicatively coupled to the wireless interface.

9. The method of claim 8, wherein the controller is configured to operate the electrothermal device in a third, thermal contrast mode in which heat energy is alternately removed and supplied to the working fluid via the heat exchanger block.

10. The method of claim 8, wherein the therapeutic component is selected from the group consisting of cooling pads, leg braces, arm/elbow braces, back braces, face pads, virtual reality headsets, comfort pads, and motorcycle helmets.

11. The method of claim 8, wherein the thermal control module is configured to be secured to at least one of: a user's arm, a user's waist, a user's leg, or a user's head.

12. The method of claim 8, wherein the electrothermal device comprises at least one Peltier component.

13. The method of claim 8, wherein the electrothermal device comprises a pair of Peltier components coupled in parallel to the heat exchanger block.

14. The method of claim 8, wherein the therapeutic component is configured to be fixed to an anatomical feature of a non-human animal.

15. A wearable, compact therapeutic thermal control system comprising:
a therapeutic component configured to be removably attached to the body of a user;
a thermal control module fluidically coupled to the therapeutic component and configured to be worn by the user, the thermal module comprising:
a power supply;
a controller;
a wireless interface communicatively coupled to the controller;
a wireless interface coupled to the controller;
a Peltier device having a first surface, a second surface, and electrical terminals communicatively coupled to the controller;
a heatsink/fan subassembly thermally coupled to the first side of Peltier device;
a heat exchanger block thermally coupled to the second side of the Peltier device; and
a pump device configured to cause flow of a working fluid through the heat exchanger block and the therapeutic component;
wherein the controller is configured to operate the Peltier device in at least two modes: a first mode in which heat energy is removed from the working fluid via the heat exchanger block, a second mode in which heat energy is supplied to the working fluid via the heat exchanger block; and a third mode in which heat energy is alternately removed and supplied to the working fluid via the heat exchanger block;
wherein the controller operates the Peltier device in response to a command received from an application running on a mobile device communicatively coupled to the wireless interface; and
wherein the therapeutic component is selected from the group consisting of cooling pads, leg braces, arm/elbow braces, back braces, face pads, virtual reality headsets, comfort pads, and motorcycle helmets.
